(19) United States
(12) Patent Application Publication
(10) Pub. No.: US 2024/0123060 A1
(43) Pub. Date: Apr. 18, 2024

(54) IMPROVED THERAPEUTIC COMPOSITION COMPRISING HEPATITIS B ANTIGEN HAVING S, PRE-S1 AND PRE-S2 PROTEIN, ALUMINIUM PHOSPHATE AND INTERFERON-ALPHA AND USE THEREOF FOR TREATMENT OF HEPATITIS B

(71) Applicants: Variation Biotechnologies Inc., Ottawa (CA); Brii Biosciences Limited, Grand Cayman (KY)

(72) Inventors: David Evander Anderson, Newton, MA (US); Zhi Hong, Chapel Hill, NC (US); Qing Zhu, Rockville, MD (US)

(21) Appl. No.: 17/621,033

(22) PCT Filed: Jun. 19, 2020
(86) PCT No.: PCT/IB2020/000539

§ 371 Date: Dec. 20, 2021

Related U.S. Application Data
(60) Provisional application No. 62/864,930, filed on Jun. 21, 2019.

(51) Int. Cl. A61K 39/29; A61K 38/21; A61K 39/39; A61P 31/20; A61P 37/04

(52) U.S. Cl. CPC A61K 39/292; A61K 38/212; A61K 39/39; A61P 31/20; A61P 37/04; A61K 2039/55505

(57) ABSTRACT

The present disclosure provides compositions and methods useful for inducing an immune response and overcoming immune tolerance in a subject suffering from Hepatitis B. As described herein, the compositions of the disclosure comprise HBsAg having S, Pre-S1 and Pre-S2 proteins, an aluminum phosphate adjuvant and interferon-α.

### CROSS REFERENCE TO RELATED APPLICATIONS

The present application claims the benefit of U.S. Prov. Appln. No. 62/864,930, filed Jun. 21, 2019, the entire contents of which are incorporated by reference herein.

### FIELD OF THE INVENTION

This invention is in the field of therapeutic compositions, in particular therapeutic compositions useful for treatment of chronic Hepatitis B infection.

### BACKGROUND

Hepatitis B is a viral infection which is the causative agent of acute and chronic liver disease. It is transmitted in humans through contact with blood or other body fluids from an infected individual. Hepatitis B infection is a widespread and significant health problem causing chronic liver disease in over 250 million people and close to a million deaths a year worldwide (Global Hepatitis Report 2017, World Health Organization). Upon infection, Hepatitis B causes an acute phase of disease, which has no symptoms in most people. Those who do show symptoms experience jaundice, fatigue and abdominal pain, with a small subset of sufferers experiencing acute liver failure which can be fatal. Following infection with Hepatitis B, a patient may either clear the virus and be cured, or he may develop a chronic Hepatitis B infection. The consequences of chronic Hepatitis B are significant, since 30% of chronically infected adults eventually develop cirrhosis and/or liver cancer. Clearance of the disease depends on a mature, healthy immune system, with infants having a 90% chance of developing a chronic disease while healthy adults only have a 5%-10% chance.

No effective treatment for Hepatitis B infection has been found. Acute infection is treated with bedrest. Chronic infection is frequently treated with antiviral agents, typically nucleoside analogs such as tenofovir, lamivudine, or entecavir. These antiviral agents can slow the progression of cirrhosis and reduce the incidence of liver cancer, but they have proven unable to achieve clearance of the virus and viral proteins continue to be produced in chronic patients. In addition to being ineffective to clear the virus, nucleoside inhibitors are costly and they must be continued for the life of the patient to maintain effectiveness.

Another common treatment for chronic infection is interferon-α, a naturally occurring cytokine produced by immune cells called dendritic cells. It has been used as a treatment for Hepatitis B since the 1970's and demonstrates advantages over nucleoside analogs including lack of drug resistance and a finite treatment course. However, interferon-α has a very short half life and must be injected multiple times a week for a period of almost a year. A pegylated form of interferon-α was introduced in 2005, which has a longer half life, allowing for one weekly subcutaneous injection over a one year period. As a result, interferon-α has been replaced by pegylated interferon-α as a standard treatment for chronic Hepatitis B because the more convenient dosing regimen results in improved patient compliance.

Treatment with interferon-α has both immunological and anti-viral properties. However, it is only effective in approximately 30% of patients (Woo et al, (2017) Ann Trans Med., 5: 159) and fewer than 10% of patients show clearance of the virus from their blood (Konerman et al, (2016) Clin Liver Dis. 20:645-655). Furthermore, interferon-α, including the pegylated version, is associated with many serious and potentially life-threatening side effects so use is frequently discontinued. Furthermore, it requires a lengthy treatment regime of injections which is difficult for some patients to accommodate or endure.

Despite decades of research, no complete cure for chronic Hepatitis B has been found. Therefore, in recent years, the focus of treatment has shifted away from complete eradication of the virus, to the achievement of a “functional cure” which has been defined as clearance of serum Hepatitis B surface antigen (HBsAg) and Hepatitis B DNA levels in which the Hepatitis B immune response is strong enough to keep viral replication under control and permit cessation of antiviral treatment (Zoulim et al. (2015) Cold Spring Harb Perspect Med 5; 5a021501). In patients, a functional cure would improve quality of life and survival rates by preventing or delaying the onset of Hepatitis B related diseases such as cirrhosis, end stage liver disease and hepatocellular carcinoma. However, achievement of a functional cure has proved elusive in chronic Hepatitis B patients, who typically show a weakened immune response to Hepatitis B viral antigens, a phenomenon referred to as “immune tolerance”. This weakened immunity is believed to be caused by high levels of Hepatitis B subviral particles which mediate Hepatitis B-specific immune dysfunction by decoying and exhausting host cellular and humoral adaptive immune responses.

Accordingly, a need exists for improved treatments for chronic Hepatitis B patients that are able to elicit a durable response in a high percentage of patients without adverse side effects and a lengthy and expensive treatment regime. In particular, a need exists for a treatment which can overcome immune tolerance in chronic Hepatitis B patients and enable the patients' own immune systems to combat the disease.

### SUMMARY

Provided are various embodiments related to therapeutic compositions for treatment of Hepatitis B comprising an immunotherapeutic component admixed with a low dose of interferon-α. The immunotherapeutic component comprises a recombinant HBsAg envelope protein comprising or consisting of S, Pre-S1 and Pre-2 protein domains (e.g., S, Pre-S1 and Pre-S2 proteins described herein) and an aluminum phosphate adjuvant. The interferon-α in the present composition is present in an amount significantly below the amount used in the standard therapeutic dose.

Further provided are uses of the therapeutic compositions of the disclosure, methods of inducing an immunological response by administering the therapeutic compositions of the disclosure, methods of overcoming immune tolerance by administering the therapeutic compositions of the disclosure and methods of treating Hepatitis B infection by administering the therapeutic compositions of the disclosure.

In some embodiments, the present disclosure provides a therapeutic composition comprising at least about 20 μg (e.g., at least about 30 μg, at least about 40 μg, at least about 50 μg, or at least about 60 μg) HBsAg envelope antigen comprising S, Pre-S1 and Pre-S2 domains, 500 μg of aluminum as aluminum phosphate adjuvant and 3 million international units (“MIU”) interferon-α. In a preferred embodiment, the present disclosure provides a therapeutic composition comprising at about least 40 μg (e.g., at least about 45%, at least about 50%, at least about 60%, at least about 70%, at least about 80%, or more) HBsAg envelope antigen comprising S, Pre-S1 and Pre-S2 domains, 500 μg of aluminum as aluminum phosphate adjuvant and 3 MIU interferon-α.

The present disclosure also encompasses the use of at least one of the therapeutic compositions of the disclosure in the preparation of a pharmaceutical composition intended for treating Hepatitis B infections.

The present disclosure further provides pharmaceutical compositions comprising the therapeutic compositions of the disclosure for administration to a subject in need thereof.

The present disclosure also provides a method for inducing a Th1 humoral and cellular immune response in a mammal comprising administering a therapeutically effective amount of a composition of the disclosure.

The present disclosure also provides a method for overcoming immune tolerance in a mammal comprising administering a therapeutically effective amount of a composition of the disclosure.

The present disclosure also provides a method for the treatment of Hepatitis B infections, in particular chronic Hepatitis B infection, comprising administering to a subject in need thereof a therapeutically effective amount of a composition of the disclosure.

Other features, objects, and advantages of the present disclosure are apparent in the detailed description that follows. It should be understood, however, that the detailed description, while indicating embodiments of the present disclosure, is given by way of illustration only, not limitation. Various changes and modifications within the scope of the disclosure will become apparent to those skilled in the art from the detailed description.

### Listing of Sequences

The following is a sequence referred to herein:

SEQ ID NO: 1 is a Large Protein of HBsAG Amino Acid Sequence

1 11 21 31
MGLSWTVPLE WGKNQSTSNP LGFFPDHQLD PAFGANSNNP
41 51 61 71
DWDLNSNKDH WPQANQVGVG AFGPGFTPPH GGLLGWSSQA
81 91 101 111
QGTLHTVPAV PPPASTNRQT KRQPTPISPP LRDSHPQAMQ
121 131 141 151
WNSTAFHQAL QHPRVRGLYF PAGGSSSGTV NPAQNIASHI
161 171 181 191
SSISSRTGDP APNMENITSG FLGPLLVLQA GFFLLTRILT
201 211 221 231
LLCLIFLLVL LDYQGMLPVC CLGQNSQSPT SNHSPTSCPP
241 251 261 271
ICPGYRWMCL RRFIIFLFIL ILLLCLIFLL VLLDYQGMLP
281 291 301 311
PLIPGSTTTS TGPCKTCTTP AQGNSMFPSC CCTKPTDGNC
321 331 341 351
TCIPIPSSWA FAKYLWEWGS VRFSWLSLLV PFVQWFVGLS
361 371 381 391
PTVWLSVIWM MWYWGPNLYN ILSPFIPLLP IFFCLWVYI

SEQ ID NO: 2 is a Small Protein of HBsAG Amino Acid Sequence

1 11 21 31
MENITSGFLG PLLVLQAGFF LLTRILTIPQ SLDSWWTSLN
41 51 61 71
FLGGSPVCLG QNSQSPTSNH SPTSCPPICP GYRWMCLRRF
81 91 101 111
IIFLFILLLC LIFLLVLLDY QGMLPVCPLI PGSTTTSTGP
121 131 141 151
CKTCTTPAQG NSMFPSCCCT KPTDGNCTCI PIPSSWAFAK
161 171 181 191
YLWEWGSVRF SWLSLLVPFV QWFVGLSPTV WLSVIWMMWY
201 211 221
WGPNLYNILS PFIPLLPIFF CLWVYI

SEQ ID NO: 3 is a Medium Protein of HBsAG Amino Acid Sequence

1 11 21 31
MQWNSTAFHQ ALQHPRVRGL YFPAGGSSSG TVNPAQNIAS
41 51 61 71
HISSISSRTG DPAPNMENIT SGFLGPLLVL QAGFFLLTRI
81 91 101 111
LTIPQSLDSW WTSLNFLGGS PVCLGQNSQS PTSNHSPTSC
121 131 141 151
PPICPGYRWM CLRRFIIFLF ILLLCLIFLL VLLDYQGMLP
161 171 181 191
VCPLIPGSTT TSTGPCKTCT TPAQGNSMFP SCCCTKPTDG
201 211 221 231
NCTCIPIPSS WAFAKYLWEW GSVRFSWLSL LVPFVQWFVG
241 251 261 271
LSPTVWLSVI WMMWYWGPNL YNILSPFIPL LPIFFCLWVY
281
I

### DETAILED DESCRIPTION OF THE EMBODIMENTS

The inventors of the present disclosure have made an improved therapeutic composition for treatment of chronic Hepatitis B which is effective at inducing Th1-type humoral and cellular immunity against Hepatitis B antigens and further, is effective to overcome immune tolerance in patients suffering from chronic Hepatitis B.

Despite decades of research, no cure for chronic Hepatitis B has been found. As a result, chronic Hepatitis B patients are subjected to ongoing treatments which, while limiting the side effects of the disease, fail, in most cases, to effect viral clearance from the blood. Complete recovery from infection with Hepatitis B depends on the ability of the patient's own immune system to clear the virus. In particular, a strong and specific T cell response is essential to achieving viral clearance of Hepatitis B (Bertoletti and Rivino, (2014) Curr Opin Infect. Dis 27:528-534). This type of response is seen almost exclusively in adult patients with mature immune systems (Bertoletti and Gehring, (2013) PLOS Path. 9:1-4). Patients with chronic Hepatitis B patients show weaker immune responses to Hepatitis B, particularly with respect to generation of virus specific cytotoxic T cells (CTLs) and T helper cells (Th). This phenomenon of impaired immune response is a well known feature of chronic Hepatitis B (Michel et al. (2011) J., Hepat. 54: 1298-1296) and is further described below. Clearance of the virus may depend on the ability of the patient's immune system to produce type 1 helper cells (“Th1 cells”), which ability appears to be impaired in chronic patients. Type 2 helper cells (“Th2 cells”) characterized by secretion of IL-4, IL-5, and IL-13 cytokines, promote antibody production and are generally associated with allergic responses and are less effective against viral infections than Th1 cells, characterized by secretion of IFN-γ. The cytokines produced by Th1 cells promote a cell mediated immune response whereas the type 2 helper cells (Th2) secrete cytokines associated with a humoral immune response.

For over 40 years, prophylactic vaccines against Hepatitis B have been available which are effective in protecting against infection and reducing the incidence of chronic Hepatitis B infections. Most of these vaccines are single antigen vaccines based on the S protein domain of the HBsAg surface antigen of Hepatitis B. The HBsAg antigen is composed of three related envelope proteins that are all encoded by the same open reading frame on the viral DNA (the “S ORF”). These three proteins have the same C terminus but differ at their N-termini due to the presence of three in-frame ATG start codons that divide the S ORF into three regions. The S or “small” envelope protein is the most abundant and the smallest with 226 amino acids (SEQ ID NO: 2). The M or “middle” surface protein includes the S protein and has an extra protein domain consisting of 55 amino acids known as pre-S2 (SEQ ID NO: 3). The protein known as the L or “large” protein consists of the S, the Pre-S2 and a third protein domain is known as pre-S1 which has 118 amino acids (SEQ ID NO: 1). However, despite their effectiveness at prophylaxis, attempts to use vaccines based on the S protein as therapeutic treatments in chronic Hepatitis B patients have proven unsuccessful (Pol et al, (2001) J. Hepatol 34: 917-921). In particular, while antibodies to HBsAg were observed in 50-85% of patients, HBsAg clearance was only rarely observed. (Michel et al. (2011) supra) Furthermore, stimulation of Th1-type immunity was not achieved (Vandpapeliere et al, (2008) Vaccine 25(51):8585-97).

The failure of prophylactic vaccines to generate an immune response in chronic patients is generally attributed to “immune tolerance”, a phenomenon whereby a Hepatitis B patient's immune system becomes unresponsive to further exposure to Hepatitis B antigens, particularly HBsAg. Immune tolerance in chronic Hepatitis B patients has been associated with the virus' large production of Hepatitis B antigens, which causes exhaustion of T cells, specifically Hepatitis B specific CD8+T cells (Boni, C. et al, (2007) J. Virol. 81: 4215-4225). Exhausted Hepatitis B-specific T cells hyper-express the programmed cell death protein 1 (PD-1) which promotes death of antigen-specific T cells. In patients with high loads of Hepatitis B virus, the destruction of antigen specific T cells can lead to the complete disappearance of these cells from the liver. The result is a weakened T cell response to stimulation by Hepatitis B antigens. The weak T cell response is not enhanced by administration of vaccines based on the S protein domain of Hepatitis B because the S antigen has a very weak ability to re-stimulate cellular immunity by a T-cell response in most subjects.

Recently, attention has focused on other HBsAg surface antigens, specifically Pre-S1 and Pre-S2, as holding potential as an immunomodulatory treatment for chronic Hepatitis B. These antigens are present at much lower levels than the S protein antigen. Furthermore, Pre-S1 mediates viral interaction with the cellular receptor for hepatocyte entry, and therefore it may represent an important target for Hepatitis B therapy. In a study using a chronic Hepatitis B mouse model, mice which demonstrated immune tolerance to the HBsAg S protein antigen were injected with a Pre-S1-polypeptide vaccine. The mice produced antibodies to Pre-S1 and mounted a Th1 cell response, which indicates that they hadn't developed immune tolerance to the Pre-S1 protein domain. Humoral and cellular immunity against Pre-S1 resulted in restoration of immunity against HBsAg, overcoming the immune tolerance that existed in the mice, and was associated with reductions in HBsAg levels and Hepatitis B DNA in serum (Bian et al, (2017) Hepat. 66:1067-1082).

In an effort to more effectively stimulate a T cell response, therapeutic vaccination studies have been carried out in chronic Hepatitis B patients using Hepatitis B vaccines which contain all three of the Pre-S1, Pre-S2 and S protein antigens. These studies showed a Hepatitis B specific T cell response in some patients. However, the effect was transient and did not lead to clearance of the disease, possibly because the vaccine stimulated a Th2 response but did not stimulate a CD8+T-lymphocyte response. (Jung et al, (2002) Vaccine 20: 3598-3612, Kosinka et al (2015) Med. Microbiol. Immunol. 204). Further attempts to induce a successful immunological response using DNA vaccines also failed to achieve a sustained response (Bertoletti and Gehring (2009) Exp. Rev, Gastro. Hep. 3: 561-569).

In a Vietnamese study, an attempt to improve responsiveness was made by combining a newer vaccine, Sci-B-Vac (a Pre-S1, Pre-S2 and S protein vaccine with aluminum hydroxide adjuvant), with a nucleoside inhibitor (lamivudine). This combination was superior to the individual therapies alone in reducing levels of viral DNA in patients. However, the effect was not sustained after the vaccine treatments were discontinued. Accordingly, this combination treatment failed to recruit the patients' immune system to effectively clear the virus (Hoa, (2009) Antimicrob. Agents and Chemo. 53: 5134-5140).

More recently, a co-inventor of the present application has found that an immunogenic composition comprising of all three HBsAg protein domains (S, Pre-S1 and Pre S-2) and an aluminum phosphate adjuvant is effective at stimulating a Th1 cell response in a mammal when the aluminum phosphate adjuvant is present in an amount that results in unbound antigen (WO2020/099927). This was an important discovery in view of previous studies which have shown no Th1 response to conventional single antigen prophylactic Hepatitis B vaccines formulated with aluminum-based adjuvants. However, the ability of this composition to stimulate a T cell response in mice declined at high doses, possibly due to the onset of a phenomenon similar to the immune tolerance observed in human chronic Hepatitis B patients. Additional stimulation of Hepatitis B CD4+ and CD8+ T cells may be required to overcome immune tolerance in order to achieve viral clearance and effect a functional cure in chronic patients.

Interferon-α has been shown to have both immunomodulatory and anti-viral effects in Hepatitis B patients. In affecting the immune system, it is known to promote both cellular and humoral immune responses. Studies have shown that interferon-α can effectively promote the differentiation and the activity of certain subsets of T and B lymphocytes and monocyte-derived dendritic cells (DC) in both mouse and human models, supporting the concept that interferon-α can act as an important factor in linking innate and adaptive immunity (Arico et al, (2012) J. Int. & Cyt. Res. 32:235-247). In Hepatitis B patients, interferon-α has been known to activate natural killer (NK) and CTLs. The immunomodulatory activity of interferon-α is highly potent but is non-specific, leading to side effects. The antiviral mechanism of interferon-α is not well understood but it is believed to act by degrading viral mRNA, inhibiting viral protein synthesis and preventing infection of cells (Rijckborst et al, (2010) Curr Hep. Rep. 9:231-238).

Since the mid-2000's, the preferred form of therapeutic interferon-α is the pegylated form, which has a longer half life and need only be administered once a week, as opposed to three times per week for standard interferon-α. Nevertheless, the duration of treatment is the same, typically 48 weeks. Treatment response is often measured by the development of antibodies to the Hepatitis B e antigen (Liaw (2009) Hepatol Int. 3:425-433). Standard treatment with pegylated interferon-α (i.e. one injection per week for 48 weeks) results in anti-Hepatitis B e seroconversion rates of approximately 30%. Rates of HBsAg loss, however, are very low at 3-7% (Zoulim (2015) supra). Resistance to interferon-α is likely caused by an inability to stimulate cellular immune responses and is associated with the chronic patient's reduced Th response to antigens of Hepatitis B virus (i.e. immune tolerance).

In addition to limited effectiveness, treatment with interferon-α, including the pegylated version, is often complicated by the occurrence of side effects. Common side effects include fatigue, headache and myalgia. In addition, neuropsychiatric side effects including depression and sleep disturbance are a significant concern, with some patients showing severe symptoms such as suicidal ideation (Rijckborst (2010) supra). As well, a smaller subset of patients show low platelet levels (thrombocytopenia) and low white blood cell count (leukopenia). As a result, discontinuation of interferon-α treatment is required in 6-9% of patients and dose modification is required in another 31-47% (Perillo et al, 2009, Heptology 49: S5). As well, due to its stimulatory effects on T cells and B cells, interferon-α has been known to exacerbate existing autoimmune disorders in patients, particularly disorders of the thyroid, rheumatoid arthritis, and psoriasis. There have also been reports of patients with no prior autoimmune conditions developing Hashimoto's thyroiditis and autoimmune hepatitis (Silva, (2012), Gastro & Hepat. 8: 540-542) as a result of interferon-α treatment. As such, while there are benefits associated with interferon-α treatment, there are significant side effects that must be monitored carefully by clinicians, and can lead to alteration or cessation of treatment.

Recent efforts to achieve a functional cure for Hepatitis B have focused on combining different treatments. Typically, interferon-α has been combined with a nucleoside analog. Nucleoside analogs reduce viral replication and therefore lower viral load in patients. Several studies have shown that treatment combining nucleoside analogs and pegylated interferon-α results in improved viral suppression during the treatment period, but the off-treatment sustained response is similar to treatment with interferon-α alone (Rijckborst (2010) supra).

Studies have also been conducted which combine interferon-α with other immunomodulatory agents, with a view to enhancing the patient's immune response to the disease. In particular, interferon-α has been tested in combination with prophylactic Hepatitis B vaccines in patients with chronic Hepatitis B.

A study to test a combination of prophylactic Hepatitis B vaccine with interferon-α in children suffering from chronic Hepatitis B found that the treated children showed a significantly lower amount of Hepatitis B DNA than the group treated with interferon-α alone. However, the results were not sustained and the difference had disappeared six months after treatment was ceased (Helvaci et al, (2004) J Gast and Hep. 19: 785-791). This study is notable because both groups received the normal, standard of care dosage of interferon-α, yet the combination group did not show a durable improvement. Furthermore, side effects typical of interferon-α treatment were present in the patients.

Another study conducted in adult patients used a combination of interferon-α at the normal, standard of care dose of 15 MIU per week and a prophylactic Hepatitis B vaccine which contained a second Hepatitis B surface antigen called Pre-S2 in addition to the S antigen. The results did not show a statistically significant difference between the group receiving the combination therapy and the group receiving only interferon-α. However, the researchers observed a different mechanism of activity, with the combination group having fewer relapses and more patients who showed a delayed response (Kaymakoglu et al (1999) AJG 94:3 856-857). It is unknown whether the different activity related to the presence of the Pre-S2 antigen or not.

None of the previous studies demonstrated that viral clearance could be achieved by combining a standard of care dose of interferon-α with a prophylactic Hepatitis B vaccine. Furthermore, these combination treatments still resulted in the side effects associated with interferon-α.

The use of lower doses of interferon-α has been investigated to determine whether it could enhance the effect of prophylactic vaccines in uninfected subjects. The importance of interferon-α for enhancing T- and B-cell functions and to act as a natural adjuvant has been recognized (Rizza et al, (2011) Expert Rev. Vaccines; 10(4); 487-498). The addition of a low dose of interferon-α (3 MIU) to a prophylactic Hepatitis B vaccine improved antibody titers in immunocompromised or non-responder populations but not in healthy subjects (Grob et al, (1984) European Journal of Clinical Microbiology; 3:195-198; Rizza et al, (2011) supra; Miquelina-Colina et al, (2009) Vaccine, 18: 5654-60). The low dose was well tolerated with only mild, transient side effects. A recent study in a recombinant mouse model using adeno-associated virus 8-1.3 Hepatitis B infection demonstrated the potential of interferon-α as an adjuvant (together with granulocyte-macrophage colony-stimulating factor) to break immune tolerance and to induce antigen-specific immune responses for an experimental Hepatitis B prophylactic vaccine (Zhao et al, (2018) Oncotarget, 9:34213-34228).

The inventors of the present invention have found that a therapeutic composition comprising a immunogenic component consisting of a Hepatitis B antigen comprising all three HBsAg protein domains (S, Pre-S1 and Pre-S2) and an aluminum phosphate adjuvant, admixed with a reduced dose of interferon-α is effective at inducing an enhanced immune response to Hepatitis B. In particular, as shown in Example 3, in a mouse model, the therapeutic composition induced increased Th1-type antibody and T cell responses against both pre-S2 and to S proteins.

Importantly, the inventors have found that the therapeutic composition of the disclosure induces a greater IgG2a response in mammals than IgG1 response. By way of contrast, the immunogenic component alone induced a higher relative IgG1 response. IgG2 is a marker for a Th1 response in mice whereas IgG1 is associated with a Th2 response. As a result, the addition of interferon-α at an adjuvant dose was sufficient to alter the profile of the immune response in the mammal to favour a Th1 response over a Th2 response. The Th1 response has been associated with clearance of Hepatitis B virus from infected patients. Therefore, the therapeutic composition of the invention, consisting of the immunogenic component admixed with a dose of interferon-α significantly below the therapeutic dose, was able to elicit an immune response in a mammal which is associated with overcoming immune tolerance and achieving viral clearance.

In a further mouse study, shown in Example 4, the inventors demonstrated that the therapeutic composition was effective to overcome immune tolerance in mice transduced to mimic chronic Hepatitis B infection. As is shown in Example 4, transduced mice showed significant reduction in T cell response to both S and pre-S1 antigens, which is typical of immune tolerance. However, when these mice were vaccinated with the therapeutic composition, the mice demonstrated a significantly higher T cell response to both S and pre-S1 proteins, indicating that the immune tolerance had been overcome.

Surprisingly, the compositions of the disclosure induced an enhanced Th1 cell response in a mammal without the presence of an adjuvant which is known to stimulate Th1-type cellular immunity, such as MPL or CpG. This is surprising in view of previous studies which have shown no Th1 response to conventional Hepatitis B vaccines.

In order to study the effect of the immunotherapeutic composition of the disclosure in human subjects with chronic Hepatitis B, a phase Ib/IIa, randomized, open-label, controlled study is being conducted to evaluate the safety, tolerability and antiviral activity of the composition in comparison to an equivalent composition without interferon-α. The compositions are administered intramuscularly to non-cirrhotic, virologically suppressed adult subjects with chronic Hepatitis B infection. This study tests two different dose levels of HBsAg envelope antigen comprising S, Pre-S1 and Pre-S2 protein domains (20 μg and 40 μg), each formulated with 500 μg of aluminum as aluminum phosphate adjuvant, with and without the addition of non-pegylated interferon-α. The study uses a dose of 3 MIU of interferon-α, which is approximately 1/10^{th} that used to treat Hepatitis B in chronic patients.

The therapeutic composition of the invention, and the immunogenic component, are administered in four single, monthly doses by intramuscular injection over a period of months for a total of four doses. This dosing schedule is considerably shorter than the standard dosing schedule for therapeutic treatment of chronic Hepatitis B with interferon-α, which is up to 48 weeks. Accordingly, the subjects will not only be administered a significantly lower dose, but they will also be exposed to interferon-α for a significantly shorter period of time.

The results of this study will indicate whether the immunogenic component of the therapeutic composition can re-stimulate a Hepatitis B-specific humoral and cellular immunity in chronic Hepatitis B patients, and whether this response is enhanced by the presence of a low dose of interferon-α. Furthermore, the results of this study will indicate whether immune tolerance can be overcome in human Hepatitis B patients, either using the immunogenic component alone, or in combination with interferon-α. By overcoming immune tolerance, these patients may be able to harness their own immune systems to clear the virus from their blood. Finally, safety observations from this study will indicate whether the low dose of interferon-α is associated with the side effects observed in standard therapeutic interferon-α treatment.

The therapeutic compositions of the disclosure comprise HBsAg which includes all three of the HBsAg antigen protein domains, specifically S, Pre-S1 and Pre-S2. In any of the aspects or embodiments described herein, a composition can comprise an S protein (e.g., an S protein comprising or consisting of an amino acid sequence having about 80%, 85%, 90%, 91%, 92%, 93%, 94%, 95%, 96%, 97%, 98%, 99%, or 100% identity to the amino acid sequence of SEQ ID NO:2); a Pre-S2 protein (e.g., a Pre-S2 protein comprising or consisting of an amino acid sequence having at least 80%, 85%, 90%, 91%, 92%, 93%, 94%, 95%, 96%, 97%, 98%, 99%, or 100% identity to residues 1 to 55 of amino acid sequence of SEQ ID NO:3); and a Pre-S1 protein (e.g., a Pre-S1 protein comprising or consisting of an amino acid sequence having at least 80%, 85%, 90%, 91%, 92%, 93%, 94%, 95%, 96%, 97%, 98%, 99%, or 100% identity to residues 1 to 118 of the amino acid sequence of SEQ ID NO:1). Embodiments described herein may comprise about 75-90% by weight S protein, about 2-8% by weight Pre-S1 protein, and about 5-15% by weight of Pre-S2 protein (e.g., 83+/−3.3% S protein, 6+/−3% Pre-S1 protein, and 11+/−3% Pre-S2 protein).

The HBsAg may originate from any genotype, strain or isolate of Hepatitis B. Further, the HBsAg may originate from a native HBsAg or from a modified HBsAg. HBsAg antigen may be isolated from a natural source of Hepatitis B virus such as biological samples (e.g. blood, plasma, sera, semen, saliva, tissue sections, biopsy specimen etc.) collected from an infected subject, cultured cells or tissue cultures. HBsAg may also be produced using recombinant techniques in cells. In some embodiments, HBsAg is expressed in a mammalian cell line. In some embodiments, HBsAg is expressed in Chinese Hamster Ovary (“CHO”) cell lines. HBsAg antigen comprising S, Pre-S1 and Pre-S2 domains may be produced using the method disclosed in U.S. Pat. No. 5,242,812. Nucleotide sequences encoding different HBsAg may be found in data banks such as Genbank and in the published literature (for example Fukimori et al (1990) 18 Nuc. Acid Res 4587; Vaudin et al (1988) 69 J. Gen Virol. 1383-1389). An amino acid sequence for the large protein of HBsAg is shown in SEQ ID NO. 1. An amino acid sequence for the small protein of HBsAg is shown in SEQ ID NO. 2. An amino acid sequence for the medium protein of HBsAg is shown in SEQ ID NO. 3.

The therapeutic compositions of the invention may comprise a single dose of 20 or 40 μg or more of HBsAg antigen having all three of the S, Pre-S1 and Pre-2 proteins. In a preferred embodiment, the therapeutic compositions of the disclosure comprise a single 40 μg dose of HBsAg antigen having all three of the S, Pre-S1 and Pre-S2 proteins.

The therapeutic compositions of the present disclosure further comprise aluminum phosphate adjuvant. One example of an aluminum phosphate adjuvant suitable for use in the present disclosure is Adju-Phos®, an aluminum phosphate wet gel suspension manufactured by Brenntag. In a preferred embodiment of the disclosure, the therapeutic composition comprises between 62.5 and 500 μg of aluminum as aluminum phosphate adjuvant. In a particularly preferred embodiment, the therapeutic composition of the disclosure comprises 500 μg of aluminum as aluminum phosphate adjuvant.

The therapeutic compositions of the present disclosure further comprise a sub-therapeutic dose of interferon-α. The therapeutic composition of the invention can comprise any form of interferon-α, and it can be unpegylated or pegylated. Preferably, the therapeutic composition of the invention comprises unpegylated interferon-α which has a shorter half life and therefore is correlated to fewer side effects in patients. One example of an interferon-α suitable for use in the present disclosure is Intron A®.

The therapeutic composition of the disclosure may comprise interferon-α in a dose of 1 MIU-6 MIU. In a preferred embodiment of the disclosure, the therapeutic composition comprises a dose of interferon-α 3 MIU units per month.

The therapeutic composition of the disclosure may comprise HBsAg antigen having all three of the S, Pre-S1 and Pre-2 proteins, aluminum phosphate adjuvant and interferon-α wherein the HBsAg antigen having all three of the S, Pre-S1 and Pre-2 proteins and interferon-α are present in a ratio of 20 to 40 μg of HBsAg antigen: 1-6 MIU of interferon-α. In a preferred embodiment of the disclosure, ratio is 40 μg of HBsAg antigen: 3 MIU of interferon-α.

The present disclosure also provides the use of a therapeutic composition of the disclosure for the preparation of a pharmaceutical composition for inducing or enhancing a Th1 immune response against Hepatitis B infection in a patient. The present disclosure also provides the use of a therapeutic composition of the disclosure for the preparation of a pharmaceutical composition for overcoming immune tolerance in a patient suffering from chronic Hepatitis B. The present disclosure further provides the use of an therapeutic composition of the disclosure for the preparation of a drug for treating Hepatitis B infection, particularly chronic Hepatitis B infection, in a patient.

The present disclosure also provides a method of inducing or enhancing a T cell immune response against Hepatitis B infection in a mammal comprising administering to the mammal a therapeutic composition of the disclosure. The immune response is preferably a Th1 response directed to a Hepatitis B antigen. Administration can be performed by injection by any means, for example by intramuscular injection. Injections can be made with conventional syringes and needles, or any other appropriate devices available in the art.

The present disclosure also provides a method for overcoming immune tolerance in a patient suffering from chronic Hepatitis B comprising administering to the patient a therapeutic composition of the disclosure. Administration can be performed by injection by any means, for example by intramuscular injection. Injections can be made with conventional syringes and needles, or any other appropriate devices available in the art.

The present disclosure also provides a method of treating Hepatitis B infection, particularly chronic Hepatitis B infection, in a subject in need thereof comprising administering a therapeutically effective amount of a composition of the disclosure. Administration can be performed by injection by any means, for example by intramuscular injection. Injections can be made with conventional syringes and needles, or any other appropriate devices available in the art.

The present disclosure also provides pharmaceutical compositions which are useful in therapeutic applications in individuals suffering from chronic Hepatitis B infection. The provided pharmaceutical compositions are formulated for delivery parenterally, e.g. by injection. In such embodiments, formulation may be suitable for intramuscular injection. Injections can be made with conventional syringes and needles, or any other appropriate devices available in the art.

In some embodiments, pharmaceutical compositions are provided in a liquid dosage form that is suitable for injection. In some embodiments, pharmaceutical compositions are provided as powders (e.g. lyophilized and/or sterilized), optionally under vacuum, which are reconstituted with an aqueous diluent (e.g., water, buffer, salt solution, etc.) prior to injection. In some embodiments, pharmaceutical compositions are diluted and/or reconstituted in water, gels, sodium chloride solution, sodium acetate solution, benzyl alcohol solution, phosphate buffered saline, etc. In some embodiments, powder should be mixed gently with the aqueous diluent (e.g., not shaken).

In some embodiments, provided pharmaceutical compositions comprise one or more pharmaceutically acceptable excipients (e.g., preservative, inert diluent, dispersing agent, surface active agent and/or emulsifier, buffering agent, etc.). Suitable excipients include, for example, water, saline, dextrose, sucrose, trehalose, glycerol, ethanol, or similar, and combinations thereof. Remington's The Science and Practice of Pharmacy, 21st Edition, A. R. Gennaro, (Lippincott, Williams & Wilkins, Baltimore, M D, 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure. In some embodiments, pharmaceutical compositions comprise one or more preservatives. In some embodiments, pharmaceutical compositions comprise no preservative.

A pharmaceutical composition in accordance with the disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredients.

Pharmaceutical compositions described herein will generally be administered in such amounts and for such a time as is necessary or sufficient to induce or enhance a response in a subject, for example an increase in anti-HBsAg antibodies or an increase in Th1 response. Dosage and duration of treatment may be varied as required to overcome immune tolerance in the patient. Dosing regimens may consist of a single dose or a plurality of doses over a period of time. In a preferred embodiment, the therapeutic compositions of the invention are administered once per month. In a particularly preferred embodiment, the therapeutic compositions of the invention are administered once per month over a four month period. The exact amount of a composition to be administered may vary from subject to subject and may depend on several factors. Thus, it will be appreciated that, in general, the precise dose used will be as determined by the prescribing physician and will depend not only on the weight of the subject, but also on the age of the subject and the severity of the symptoms.

The invention has been described in an illustrative manner and many modifications and variations of the present invention are possible in light of the above teachings. It is therefore to be understood that within the scope of the claims, the invention may be practiced in a different way from what is specifically described herein.

All of the above cited disclosures of patents, publications and database entries are specifically incorporated herein by reference in their entirety to the same extent as if each such individual patent, publication or entry were specifically and individually indicated to be incorporated by reference.

### EXAMPLES

The following examples describe some exemplary modes of making and practicing certain compositions that are described herein. It should be understood that these examples are for illustrative purposes only and are not meant to limit the scope of the compositions and methods described herein.

### Example 1: Preparation of Vaccine Formulations Containing Aluminum Phosphate Adjuvant, with and without Mouse Interferon-α

A Hepatitis B surface antigen consisting of all three of the S, pre-S1 and pre-S2 proteins was prepared in CHO cells in accordance with the method described in U.S. Pat. No. 5,242,812.

Vaccine formulations comprising aluminum phosphate adjuvant (Adjuphos®) were prepared as follows. Briefly, three different concentrations of Hepatitis B surface antigen described above (20 μg/ml, 40 μg/ml and 60 μg/ml) using 500 μg/ml of aluminum content of aluminum phosphate adjuvant (Adjuphos) were rotated at 15±5 rpm for 60 minutes at room temperature. More specifically, Adjuphos adjuvant was added to a sterile container followed by a 10 mM phosphate 8% sucrose buffer and PBS. Bulk HBsAg antigen was added to the same container and mixed slowly by aspirating pipette. The container was sealed and covered with aluminum foil, and then rotated for 60 minutes at 8-12 rpm at room temperature. Once rotation was completed, the vaccine formulation was stored at 2-8° C. until admixed with commercially available interferon-α at a concentration of 6 MIU/ml.

For the compositions with interferon-α, one vial of liquid interferon-α (Biolegend: 10 μ/mL of interferon-α) was rested at room temperature for 15-30 minutes. Using a sterile 1 mL syringe and 27G ½ needle, 0.55 ml of interferon-α was added to an empty sterile 2 ml glass vial. One vial of antigen composition was rested at room temperature for 15-30 minutes and 0.55 ml of the antigen composition is added to the 0.55 ml of interferon-α and swirled for 20-30 seconds. The composition must be administered within one hour of preparation if kept at room temperature or within 3 hours if refrigerated at 2-8° C.

### Example 2: Evaluation of Antibody Immune Responses in Mice after Vaccination with Different Therapeutic Compositions Comprising HBsAg Comprising S, Pre-S1 and Pre-S2, Interferon-α and Aluminum Phosphate Antigen

This example describes evaluation of the immune response in mice following immunization with different therapeutic formulations comprising different concentrations of antigen (HBsAg comprising S, Pre-S1 and Pre-S2 protein), 500 μg/ml of aluminum from aluminum phosphate adjuvant and interferon-α.

Female Balb/C matched for age (6-8 weeks old) and weight (16-24 g) were used for this study. Mice were randomly assigned to 4 experimental groups with 8 animals each in accordance with the study design shown in Table 1.

TABLE 1

| | HBsAg | HBsAG | Al+++ | Al+++ | Mouse IFNα | Mouse IFNα |
|---|---|---|---|---|---|---|
| Group | (μg/ml) | (μg/dose) | (μg/ml) | μg/dose | IU/ml | IU/dose |
| 1 | 20 | 1 | AlPO4: 500 | 25 | — | — |
| 2 | 20 | 1 | AlPO4: 500 | 25 | 200000 (10 μg/mL) | 10000 (0.5 μg) |
| 3 | 40 | 2 | AlPO4: 500 | 25 | 200000 (10 μg/mL) | 10000 (0.5 μg) |
| 4 | 60 | 3 | AlPO4: 500 | 25 | 200000 (10 μg/mL) | 10000 (0.5 μg) |

Balb/c mice were vaccinated 3 times on weeks 0, 3, 6. Mice were sacrificed on day 6 post 3^{rd} vaccination to measure immune responses to the pre-S1, pre-S2, and HBsAg S proteins using overlapping peptide pools and anti-HBsAg IgG1/IgG2a were measured using serum samples.

Antibodies to HBsAg were measuring by ELISA using commercially available kits as follows. Well plates were coated overnight at 4° C., with recombinant Hepatitis B Surface antigen Protein, Abcam (0.25 μg/ml in DPBS). The following day, plates were blocked with 10% goat sera in ELISA wash buffer, for 1 hour at 37° C. Plates were washed with wash buffer, followed by addition of 2 fold dilutions, of individual mouse sera; starting at 1:20,000 to 1:2,560,000. Plates were incubated, for 1.5 hours at 37° C., followed by plate washing and addition of Secondary Antibody: (i) Goat anti-Mouse IgG1 (Bethyl) or (ii) Goat anti-Mouse IgG2a (Bethyl) or (iii) Goat anti-Mouse Total IgG (Bethyl) or (iv) Goat anti-Mouse Total IgG (Bethyl), diluted 1:10,000 in 10% goat sera in ELISA wash buffer, plates were incubated for 1.5 hours at 37° C. Plates were added with TMB One component Microwell substrate, incubated at room temperature for 10 minutes and then added with Stop solution. Absorbance was read at 450 nm using a MAXline plate reader.

The results are shown in Table 2 below. What is reported is the ratio of the geometric mean titer of IgG1:IgG2a antibodies against HBsAg. Lower ratios demonstrate a qualitative Th1-type change in the nature of the antibody response. Formulation with IFN-α enhanced the Th1-type antibody response, which was particularly evident with the higher dose formulation (Group 4).

TABLE 2

| Mouse # | Group 1 | Group 2 | Group 3 | Group 4 |
|---|---|---|---|---|
| 1 | 3.9 | 4.3 | 1.5 | 0.4 |
| 2 | 0.5 | 3.6 | 8.9 | 1 |
| 3 | 4.8 | 2.4 | 7 | 1.8 |
| 4 | 1.1 | 2.1 | 0.2 | 0.7 |
| 5 | 5.9 | 2.9 | 2.8 | 0.3 |
| 6 | 62.4 | 1.3 | 0.7 | 0.02 |
| 7 | 5.6 | 0.1 | 5.2 | 0.4 |
| 8 | 0.9 | 0.2 | 0.4 | 1.1 |
| Mean | 10.64 | 2.113 | 3.338 | 0.715 |

### Example 3: Evaluation of Cellular Immune Responses in Mice after Vaccination with Different Therapeutic Compositions Comprising HBsAg Comprising S, Pre-S1 and Pre-S2, Interferon-α and Aluminum Phosphate Adjuvant

Female Balb/C matched for age (6-8 weeks old) and weight (16-24 g) were used for this study. Mice were randomly assigned to 6 experimental groups with 8 animals each in accordance with the study design shown in Table 3.

TABLE 3

| Exp. | HBsAg | HBsAG | Al+++ | Al+++ | Mouse IFNα | Mouse IFNα*** |
|---|---|---|---|---|---|---|
| Group | (μg/mL) | (μg/dose) | (μg/mL) | μg/dose | IU/mL | IU/dose |
| 1 | 20 | 1 | AlPO4: 500 | 25 | — | — |
| 2 | 20 | 1 | AlPO4: 500 | 25 | 200000 (10 μg/mL) | 10000 (0.5 μg) |
| 3 | 40 | 2 | AlPO4: 500 | 25 | | |
| 4 | 40 | 2 | AlPO4: 500 | 25 | 200000 (10 μg/mL) | 10000 (0.5 μg) |
| 5 | 60 | 3 | AlPO4 | 25 | | |
| 6 | 60 | 4 | AlPO4: 500 | 25 | 200000 (10 μg/mL) | 10000 (0.5 μg) |

Balb/c mice were vaccinated 3 times on week 0, 3, 6. Mice were sacrificed on day 6 post 3^{rd} vaccination to measure immune responses to the pre-S1, pre-S2, and HBsAg S proteins using overlapping peptide pools by enzyme linked immunospot assay (“ELISPOT”).

IFN-γ ELISPOT analyses to measure Th1 T cell responses were performed as follows. After sacrifice, spleens were removed and processed to produce single cell suspensions. Erythrocytes were lysed using a commercially available buffer (BioLegend). Splenocytes were then re-suspended at 6×10⁶ splenocytes/mL. On the day of spleen collection and processing, the coated ELISPOT plates were washed 5 times with 200 μl sterile PBS and blocked with 100 μl of R10 media for 1-2 hrs. Once the splenocytes had been isolated and counted, the R10 blocking media was removed and 50 μl of splenocytes (300,000 cells) and 50 μl of the stimulants were plated onto the ELISPOT assay plates. Splenocytes from each mouse were stimulated in the presence of 0.1 ng/ml rmIL-2 in duplicate with following stimulants: Pre-S1 (final stimulation concentration=27 μg/ml), Pre-S2 (final stimulation concentration=11 μg/ml) and HBsAg (final stimulation concentration=54 μg/ml), R10+DMSO as a negative control and phorbol 12-myristate 13-acetate and ionomycin (PMA (20 ng/ml)/Ionomycin (1 μg/ml) as a positive control. The ELISPOT plates were then placed into a humid 37° C. with 5% CO2 incubator for 40-48 hours. After incubation, the plates were washed 5 times with 200 μl PBS-Tween for removal of splenocytes, stimulants and media and 100 μl of IFN-γ capture antibody (Mabtech) at a concentration of 1 μg/ml was then added to each well. Following a 2 hour incubation, the ELISPOT plates were washed 5 times with PBS-Tween and 100 μl streptomycin horseradish peroxidase (strep-HRP) diluted 1:1000 was added to each well. The plates were then incubated for a further hour before being developed for 30 minutes at room temperature by adding 100 μl 3-Amino-9-ethylcarbazole (AEC) substrate (BD BioSciences). The observed spots were counted by ZellNet Consulting and the final data reported as spot forming units (SFC) per one million splenocytes.

The results for the HBsAg ELISPOT are shown in Table 4

TABLE 4

| Mouse | Group 1 | Group 2 | Group 3 | Group 4 | Group 5 | Group 6 |
|---|---|---|---|---|---|---|
| 1 | 344.655 | 1122.21 | 346.32 | 456.21 | 532.8 | 692.64 |
| 2 | 139.86 | 452.88 | 569.43 | 670.995 | 266.4 | 754.245 |
| 3 | 344.655 | 288.045 | 614.385 | 699.3 | 944.055 | 662.67 |
| 4 | 531.135 | 308.025 | 281.385 | 258.075 | 318.015 | 712.62 |
| 5 | 379.62 | 296.37 | 787.545 | 1108.89 | 151.515 | 804.195 |
| 6 | 862.47 | 705.96 | 840.825 | 1153.845 | 509.49 | 1883.115 |
| 7 | 1182.15 | 158.175 | 1107.225 | 1120.545 | 231.435 | 1030.635 |
| 8 | 199.8 | 536.13 | | 884.115 | | 1142.19 |
| Mean | 498 | 483.5 | 649.6 | 794 | 422 | 960.3 |

The results for the Pre-S2 ELISPOT are shown in Table 5

TABLE 5

| Mouse | Group 1 | Group 2 | Group 3 | Group 4 | Group 5 | Group 6 |
|---|---|---|---|---|---|---|
| 1 | 101.565 | 790.875 | 28.305 | 139.86 | 149.85 | 81.585 |
| 2 | 28.305 | 308.025 | 259.74 | 204.795 | 106.56 | 103.23 |
| 3 | 174.825 | 76.59 | 394.605 | 326.34 | 153.18 | 91.575 |
| 4 | 226.44 | 174.825 | 21.645 | 76.59 | 93.24 | 367.965 |
| 5 | 261.405 | 228.105 | 111.555 | 198.135 | 73.26 | 73.26 |
| 6 | 357.975 | 489.51 | 504.495 | 389.61 | 321.345 | 979.02 |
| 7 | 599.4 | 71.595 | 238.095 | 589.41 | 144.855 | 451.215 |
| 8 | 339.66 | 313.02 | | 274.725 | | 727.605 |
| Mean | 261.2 | 306.6 | 222.6 | 274.9 | 148.9 | 359.4 |

These data indicate that the novel composition comprising a very low dose of interferon-α, admixed with the immunogenic component consisting of the Hepatitis B S, Pre-S1 and Pre-S2 antigen with an aluminum phosphate adjuvant was able to stimulate a Th1 response in a mammal model. Surprisingly, at the highest dose of immunogenic formulation tested which lacked interferon-α, there was a reduction in the Th1 T cell responses, particularly against HBsAg. This is evidence of peripheral T cell tolerance often observed when stimulating the immune system with very high doses of antigen, and is reminiscent of the tolerance observed in patients chronically infected with Hepatitis B. In marked contrast, there was no reduction, in fact an increase, in the Th1 T cell responses observed with the highest dose of immunogenic formulation formulated with interferon-α. Accordingly, it appears that the addition of interferon-α at a low dose was successful in preventing immune tolerance and facilitating an enhanced Th1 response.

### Example 4: Evaluation of Cellular and Antibody Immune Responses in Transduced Mice after Vaccination with Different Therapeutic Compositions Comprising HBsAg Comprising S, Pre-S1 and Pre-S2, and Aluminum Phosphate Adjuvant, with and without Interferon-α

Male C57Bl/6 mice were randomly assigned to 4 experimental groups with 4 animals each. Two groups were transduced with a recombinant adenoviral vector (rAAV 8-1.3, 1×10¹¹ genome copies/mouse, genotype D) expressing the Hepatitis B genome (“ADV-HBV”) in order to mimic chronic Hepatitis B infection in mice, using the technique described in Yang et al, (2014) Cell & Mol Bio 11, 71-78. Approximately 4 months after transduction with ADV-HBV, which was sufficient time for immune tolerance of the Hepatitis B T cell response to occur, mice were vaccinated intramuscularly with one of formulation i) a composition comprising 1 μg of HBsAg comprising S, Pre-S1 and Pre-S2 protein and 0.5 μg of aluminum from aluminum phosphate adjuvant or formulation ii) a composition comprising 1 μg of HBsAg comprising S, Pre-S1 and Pre-S2 protein and 0.5 μg of aluminum from aluminum phosphate adjuvant co-formulated with murine interferon-α (0.5 μg) on days 1, 21, and 42. Responses were compared to those induced in mice which had not been transduced with AAV-HBV.

Livers of the mice were collected one week after the final vaccination, processed using a standard Ficoll/Percol gradient methodology to obtained infiltrating T cells, and combined for each group in order to ensure sufficient numbers were available for use in an IFN-γ-secreting ELISPOT assay (Mabtech-3321-4AST-2) performed as described in Example 3. Equivalent numbers of cells (2×10⁵) were stimulated with overlapping peptide pools (1 μg/ml) spanning the HBsAg or pre-S1 antigens, or with media alone, for 18 hours. The spot number was reported for each condition (each represents an antigen-specific T cell evidenced by secreting of IFN-γ), as was the activity (which represents the area of each spot rather than just the number spots). The results are summarized in Table 6.

TABLE 6

| | | Spot number | | | Activity | | |
|---|---|---|---|---|---|---|---|
| | AAV/HBV | Media | | Pre | Media | | Pre |
| Group | status | alone | HBsAg | S1 | alone | HBsAg | S1 |
| Group 1 | None | 180 | 421 | 517 | 2467 | 10894 | 12603 |
| HBsAg/AlPO4 1 ug/mouse, IM, on | | | | | | | |
| days 1, 21 and 42 | | | | | | | |
| Group 2 | | 189 | 1335 | 1601 | 2912 | 26123 | 31760 |
| HBsAg/AlPO4 & IFNα 1 ug/mouse & | | | | | | | |
| 0.5 ug/mouse, IM, on days 1, 21 and 42 | | | | | | | |
| Group 3 | AAV-HBV | 45 | 110 | 69 | 538 | 1668 | 1150 |
| HBsAg/AlPO4 1 ug/mouse, IM, on | transduced | | | | | | |
| days 1, 21 and 42 | | | | | | | |
| Group 4 | | 84 | 457 | 866 | 894 | 8248 | 18220 |
| HBsAg/AlPO4 & IFNα 1 ug/mouse & | | | | | | | |
| 0.5 ug/mouse, IM, on days 1, 21 and 42 | | | | | | | |

As shown above in Table 6, the non-transduced mice injected with formulation i) and ii) showed a detectable T cell response against both the HBsAg and PreS1 antigens, which response was significantly enhanced in mice which were injected with formulation ii) containing IFN-γ (Group 2 vs 1). There was a dramatic loss of T cell responses to both HBsAg and PreS1 antigens in AAV-HBV transduced mice in mice vaccinated with both formulation i) and formulation ii). This reduced response demonstrates immune tolerance. However, T cell responses were four times higher in AAV-HBV-transduced mice vaccinated with formulation ii) which includes IFN-α than with formulation i) (Group 4 vs. 3). These data demonstrate that a formulation comprising HBsAg comprising S, Pre-S1 and Pre-S2, and aluminum phosphate antigen, with IFN-α was uniquely able to overcome immune tolerance in a mouse model of chronic Hepatitis B infection.

Antibodies to HBsAg were measured after day 42 by ELISA using commercially available Hepatitis B Anti-HBs kits (Autobio, CL 0311) as follows. 50 μl of five standards (1000 mIU/mL, 300 mIU/mL, 90 mIU/mL, 25 mIU/mL and 5 mIU/mL), diluted serum samples (600 fold), DPBS (negative control) were added to the reaction wells of an HBsAg coated microtiter plate. 50 μl Anti-HBs conjugate reagent (horseradish peroxidase labeled with HBsAg in stability buffer) was added into each reaction well. Plates were covered and incubated for 1.0 hour at 37° C., followed by washing five times with 350 μl of wash buffer. After washing, 50 μl of a mixture of reagent A and B were added to each reaction well. The wells were covered and incubated at room temperature for 10 minutes. Luminescence was read immediately at using a BioTek Synergy 2 plate reader.

The results are shown in Table 7 below.

TABLE 7

| | Group 1 | Group 2 | Group 3 | Group 4 |
|---|---|---|---|---|
| | Anti-HBs | Anti-HBs | Anti-HBs | Anti-HBs |
| Mouse | content | content | content | content |
| number | (mIU/ml) | (mIU/ml) | (mIU/ml) | (mIU/ml) |
| 1 | 4056.41 | 9778.94 | 14.41 | 318.61 |
| 2 | 79.36 | 34.72 | 12.29 | 419.83 |
| 3 | 159.59 | 2884.57 | 54.53 | 20.59 |
| 4 | 91.82 | 1669.54 | 13.83 | 100.04 |
| average | 1096.79 | 3591.94 | 23.77 | 214.77 |

As shown in Table 7, formulation ii), which contains IFN-α, induced a much stronger antibody response in mice to HBsAg. This enhancement was observed in the mice transduced with AAV-HBV which were immune tolerance. Therefore, antibody responses also demonstrated that formulation ii) was able to overcome immune tolerance in mice.

### Other Embodiments

Other embodiments of the disclosure will be apparent to those skilled in the art from a consideration of the specification or practice of the disclosure disclosed herein. It is intended that the specification and examples be considered as exemplary only, with the true scope of the disclosure being indicated by the following claims. The contents of any reference that is referred to herein are hereby incorporated by reference in their entirety.

What is claimed is:

1. A therapeutic composition comprising:
(a) an HBsAg antigen comprising S protein, Pre-S1 protein and Pre-S2 protein;
(b) an aluminum phosphate adjuvant; and
(b) interferon-α,
wherein the composition comprises at least 20 μg of the HBsAg antigen and 1 MIU-6 MIU of the interferon-α.

2. The therapeutic composition of claim 1 wherein the HBsAg antigen has an amino acid sequence at least 90% sequence identity to SEQ: ID NO 1.

3. The therapeutic composition of claim 1 wherein the HBsAg antigen has an amino acid sequence at least 95% sequence identity to SEQ: ID NO 1.

4. The therapeutic composition of claim 1 wherein the HBsAg antigen has an amino acid sequence of SEQ: ID NO 1.

5. The therapeutic composition of any one of claims 1 to 4 wherein the composition comprises 3 MIU of the interferon-α.

6. The therapeutic composition of any one of claims 1 to 5 wherein aluminum phosphate adjuvant is present in an amount of 500 μg.

7. The therapeutic composition of any one of claims 1 to 6 wherein the composition comprises 40 μg of the HBsAg antigen.

8. The therapeutic composition of claim 1 wherein the HBsAg antigen and interferon-α are present in a ratio of at least 20 μg of HBsAg antigen: 3 MIU interferon-α.

9. The therapeutic composition of claim 1 wherein the HBsAg antigen and interferon-α are present in a ratio of 20 to 40 μg of HBsAg antigen: 1-6 MIU of interferon-α.

10. The therapeutic composition of claim 1 wherein the HBsAg antigen and interferon-α are present in a ratio of 40 μg of HBsAg antigen: 3 MIU of interferon-α.

11. The therapeutic composition of claim 1 for use inducing a Th1 cell response in a mammal.

12. The therapeutic composition of claim 1 for use in overcoming immune tolerance in a subject suffering from chronic Hepatitis B.

13. Use of the therapeutic composition of claim 1 for manufacture of a drug for inducing a Th1 cell response in a subject.

14. The use of claim 13 wherein the subject has been infected with Hepatitis B.

15. Use of the therapeutic composition of claim 1 for manufacture of a drug for overcoming immune tolerance in a subject suffering from chronic Hepatitis B.

16. A pharmaceutical composition comprising the therapeutic composition of any one of claims 1 to 11 and a pharmaceutically acceptable excipient.

17. The pharmaceutical composition of claim 11 for use in treating a subject suffering from Hepatitis B.

18. A method of inducing a Th1 cell response in a mammal, said method comprising administering the therapeutic composition of claim 1.

19. A method of overcoming immune tolerance in a subject suffering from chronic Hepatitis B, said method comprising administering the therapeutic composition of any one of claims 1 to 12.

20. A method of treating Hepatitis B in a subject, said method comprising administering a therapeutically effective amount of the therapeutic composition of any one of claims 1 to 12 to the subject.

21. The method of claim 20 wherein an additional Hepatitis B treatment is administered to the subject prior to, concurrently with, or after administration of the therapeutic composition to the subject.

22. The method of claim 21 wherein the additional Hepatitis B treatment is a polymerase inhibitor, an RNase H inhibitor, a TLR agonist, an N-glycosylation inhibitor, an antisense oligonucleotide, an anti-hepatitis B antibody, a capsid inhibitor, a core protein inhibitor, a core assembly modulator, an S-antigen reducer or sequesterer, a nucleic acid polymers, a ccc DNA inhibitor, or an siRNA.

23. A method of treating Hepatitis B in a subject, said method comprising combining an immunogenic composition comprising:
(i) an HBsAg antigen comprising S protein, Pre-S1 protein and Pre-S2 protein; and
(ii) an aluminum phosphate adjuvant,
with interferon-α to form an admixture, wherein the admixture comprises at least 20 μg of HBsAg antigen and 1 MIU-6 MIU of interferon-α and administering the admixture to a subject in need thereof.


SEQUENCE LISTING (3 sequences)

SEQ ID NO: 1 - protein, 399 aa, Hepatitis B virus
MGLSWTVPLEWGKNQSTSNPLGFFPDHQLDPAFGANSNNPDWDLNSNKDHWPQANQVGVGAFGPGFTPPHGGLLGWSSQAQGTLHTVPAVPPPASTNRQTKRQPTPISPPLRDSHPQAMQWNSTAFHQALQHPRVRGLYFPAGGSSSGTVNPAQNIASHISSISSRTGDPAPNMENITSGFLGPLLVLQAGFFLLTRILTLLCLIFLLVLLDYQGMLPVCCLGQNSQSPTSNHSPTSCPPICPGYRWMCLRRFIIFLFILILLLCLIFLLVLLDYQGMLPPLIPGSTTTSTGPCKTCTTPAQGNSMFPSCCCTKPTDGNCTCIPIPSSWAFAKYLWEWGSVRFSWLSLLVPFVQWFVGLSPTVWLSVIWMMWYWGPNLYNILSPFIPLLPIFFCLWVYI

SEQ ID NO: 2 - protein, 226 aa, Hepatitis B virus
MENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTSTGPCKTCTTPAQGNSMFPSCCCTKPTDGNCTCIPIPSSWAFAKYLWEWGSVRFSWLSLLVPFVQWFVGLSPTVWLSVIWMMWYWGPNLYNILSPFIPLLPIFFCLWVYI

SEQ ID NO: 3 - protein, 281 aa, Hepatitis B virus
MQWNSTAFHQALQHPRVRGLYFPAGGSSSGTVNPAQNIASHISSISSRTGDPAPNMENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTSTGPCKTCTTPAQGNSMFPSCCCTKPTDGNCTCIPIPSSWAFAKYLWEWGSVRFSWLSLLVPFVQWFVGLSPTVWLSVIWMMWYWGPNLYNILSPFIPLLPIFFCLWVYI